(19) United States
(12) Patent Application Publication
(10) Pub. No.: US 2012/0264114 A1
(43) Pub. Date: Oct. 18, 2012

(54) Disposable Device for the Detection of Particles of Interest, Such as Biological Entities, Detection System Comprising Said Device and Method for Using Same

(75) Inventors: Bruno François Marcel Wacogne, Traitiefontaine (FR); Christian Gérard Daniel Pieralli, Besancon (FR); Wilfrid Hervé Boireau, Mondon (FR); Thomas Mangeat, Besançon (FR); Alain Léon René Coaquette, Salans (FR); Lionel Pazart, Besancon (FR); Christian Louis Davrinche, Montaigut Sur Save (FR); Serge Piranda, Besancon (FR); Jean-François Maurice Louis Delforge, Auxon Dessous (FR)

(73) Assignee: CENTRE NATIONAL DE LA RECHERCHE SCIENTIFIQUE, Paris (FR)

(21) Appl. No.: 13/499,219

(22) PCT Filed: Sep. 28, 2010
(86) PCT No.: PCT/FR2010/000643

§ 371 Date: Jun. 29, 2012

(30) Foreign Application Priority Data
Sep. 29, 2009 (FR) 09 04633

(51) Int. Cl. G01N 33/569; G01N 33/543; G01N 21/64

(52) U.S. Cl. 435/5; 435/7.1; 436/518; 422/69

(57) ABSTRACT

The invention relates to a disposable device comprising all of the reagents necessary for the detection of one or more particles of interest, and which can be incorporated in a detection system including simple means for the automatic management of fluids. For this purpose, the invention relates to a disposable device for the detection of one or more particles of interest present in a liquid sample, said device comprising a substrate provided with: a chamber for capturing the particle(s) of interest to be detected; a fluid channel connecting, upstream, the capture chamber to a buffer solution container, a liquid sample injection means and a container of labelling probes that can be secured to the particle(s) of interest to be detected; and a fluid channel connecting, downstream, the capture chamber to a container for the recovery of liquids which, during use, can flow from the capture chamber.

The invention relates to a disposable device for the detection of particles of interest, such as biological entities, to a detection system comprising said device and to a method for using same.

The disposable device comprises a part specific to the biological entity to be detected.

The detection system comprises a detection part that can be adapted to the type of disposable device used.

Currently, the development of embedded systems, allowing fast detection of particles of interest such as pathogenic agents or other biological entities, is in a phase of rapid expansion.

An embedded device is an autonomous device which performs a predefined task. It must be easy to transport, and consume sufficiently little energy to be able to operate on an autonomous battery, and it must have the shortest possible response time.

In addition, its use must be economical while at the same time providing irreproachable hygiene and reliable results.

Currently, the detection of biological entities (mainly proteins and viruses) is carried out by means of bulky devices that have to be located in specific laboratories. This detection takes several days and makes certain tasks very restricting.

The techniques mainly used are ELISA (Enzyme-liked immunosorbent assay), PCR (polymerase chain reaction) or immuno-PCR and Biacore-type plasmonic resonance systems.

These systems are bulky and/or expensive and they could in no way be used portably. Nevertheless, some attempts at miniaturization have been proposed.

Among these tests, mention may be made of the system developed by Ymeti and colleagues (A. YMETI, J. GREVE, P. V. LAMBECK et al., “Fast, ultrasensitive virus detection using a Young interferometer sensor”. Nano letters [online], 2006, vol. 0, No. 0, pp A-D).

This system is based on the phenomenon of interference of two light rays resulting from the same source (monochromatic laser).

The device described in this article comprises at least two waveguides. Antibodies complementary to the virus to be detected are arranged at the surface of one of the guides. Then, the two light beams are sent down the waveguides. One of the guides is not modified by the presence of virus, while, in the other, the presence of viruses bound to the antibodies slightly modifies the light propagation speed. The interferences at the output are then imaged on a camera and then analyzed (Fourier transform processing).

This device is rapid, sensitive and easy to use. It also has the advantage of not requiring the labeling of molecules.

However, the interferometric technique proposed is based on the preparation of optical waveguides, the manufacture of which is difficult and expensive (clean-room microtechnology). Furthermore, the analysis of the interferometric signal recorded on the CCD sensor of the camera can be extremely difficult in the case of a simultaneous multidetection system. Finally, no mention is made of the management of the various fluids necessary for the detection.

This article therefore describes an outline device which is expensive and incompatible with the requirements of an effective embedded device.

The objective of the present invention is therefore to propose a reliable, economical embedded device which allows rapid and precise detection, without requiring specific technical knowledge on the part of the user.

For this, the invention proposes producing a device that is sufficiently economical to be disposable, comprising all of the reagents necessary for the detection of one or more particles of interest, and which can be integrated into a detection system incorporating simple means for the automatic management of fluids.

To this end, the subject of the invention is a disposable device for the detection of one or more particles of interest present in a liquid sample, said device comprising a substrate provided with:
a chamber for capturing the particle(s) of interest to be detected;
a fluidic channel connecting, upstream relative to the direction of flow during use, the capture chamber:
   to a container pre-filled with a predefined volume of buffer solution;
   to a liquid sample injection means;
   to a container pre-filled with a predefined volume of labeling probes capable of binding to the particle(s) of interest to be detected; and
a fluidic channel connecting, downstream relative to the direction of flow during use, the capture chamber to a container for the recovery of the liquids that may flow, during use, from the capture chamber.

By virtue of the embedded system according to the invention, the disposable nature makes it possible to ensure irreproachable hygiene and reliable results. In addition, the automatic management of the fluids pre-integrated in the device allows rapid use, including by unspecialized personnel.

The device and the system subsequently described can be used for any pathogenic agent bound to a surface via a ligand-bound specific linker and revealed by binding of a probe which allows a measurement (optical, magnetic, etc.) to be made. For each agent, it will be advisable to adjust the system (type of fluid, type of probe(s), fluid volumes, fluid management, type of detection, ligand-bound, etc.).

According to other embodiments:
the substrate may also comprise a liquid-sample container placed downstream of the sample injection means and upstream of the capture chamber;
the buffer solution container and the labeling probe container can have a pressurization structure designed to allow the flow, during use, of the buffer solution and of the labeling probes to the capture chamber via the upstream fluidic channel;
the sample container can have a pressurization structure designed to allow the flow, during use, of the sample to the capture chamber via the upstream fluidic channel;
the buffer solution container and the labeling probe container can be made of a material that is sufficiently flexible to be deformed by an external pressure, between a storage volume and an ejection volume;
the sample container can be made of a material that is sufficiently flexible to be deformed by an external pressure, between a storage volume and an ejection volume;
the buffer solution container and the labeling probe container can be made of a material that is sufficiently rigid to allow, during use, an internal raised pressure that is sufficient to drive respectively the buffer solution and the labeling probes to the capture chamber;
the sample container can be made of a material that is sufficiently rigid to allow, during use, an internal raised pressure that is sufficient to drive the sample to the capture chamber;
the sufficiently rigid material may comprise a membrane made of a leaktight material that retains its leaktightness after having been pierced, preferably chosen from a silicone polymer, such as polydimethylsiloxane (PDMS), poly(methyl methacrylate) (PMMA), polyvinyl chloride (PVC) and Tygon®;
the liquid sample injection means may comprise a membrane made of a leaktight material that retains its leaktightness after having been pierced, preferably chosen from a silicone polymer, such as polydimethylsiloxane (PDMS), poly(methyl methacrylate) (PMMA), polyvinyl chloride (PVC) and Tygon®;
the buffer solution container, the sample container and the labeling probe container can have a pressure-sensitive non-return valve allowing, during use, opening of the valve when a threshold pressure is applied;
the recovery container can have a volume at least equal to the sum of the volumes of the buffer solution container, of the secondary container of the liquid sample and of the labeling probe container;
the capture chamber can have:
   at least one surface functionalized with ligands, bound to said surface, and capable of associating specifically with the particle(s) of interest to be detected; and
   at least one surface designed to allow, during use, the detection of the labeling probes bound to the particle(s) of interest which have associated specifically with the ligands;
the substrate can also comprise at least one liquid sample reagent container, placed downstream of the sample injection means and upstream of the capture chamber;
the substrate can also comprise at least one reagent container, placed downstream of the liquid sample container and upstream of the capture chamber; and/or
the device can also comprise a suction channel which opens into the liquid sample container and which can be connected to a pump for sucking the liquid sample from the injection means to the liquid sample container.

The invention also relates to a system for the detection of one or more particles of interest present in a liquid sample, said system being provided with a housing comprising:
a receptacle designed to receive an above disposable device;
a means for pressurization of the container pre-filled with a predefined volume of buffer solution and of the container pre-filled with a predefined volume of labeling probes; and
a labeling probe detection means.

According to other embodiments:
the detection system can also comprise a means for pressurization of the sample container;
the detection system can comprise a receptacle designed to receive an above disposable device, and in which the pressurization means is a mechanical, electromechanical, pneumatic or hydraulic device designed to deform the containers;
the detection system can comprise a receptacle designed to receive an above disposable device, and in which the pressurization means comprises a means for injecting a fluid under pressure into the containers; and/or
the detection system can also comprise a removable cassette designed to receive the substrate of an above disposable device, and intended to be inserted into the receptacle.

The invention also relates to a method for using an above disposable device, comprising the following steps:
a) injecting the sample to the capture chamber, in order to trap the particles of interest to be detected;
b) evacuating the sample to the liquid recovery container;
c) causing the buffer solution to flow from its container to the capture chamber, in order to rinse the untrapped particles from said chamber;
d) evacuating the buffer solution to the liquid recovery container;
e) causing the probes to flow from their container to the capture chamber, so that they bind to the particles of interest trapped in the capture chamber;
f) evacuating the probes to the liquid recovery container;
g) causing the buffer solution to flow from its container to the capture chamber, in order to rinse the unbound probes from said chamber;
h) evacuating the buffer solution to the liquid recovery container;
i) measuring the presence or absence of the probes, and therefore of the particles of interest to be detected.

According to other embodiments:
steps a) to d) can be carried out sequentially until all of the sample has been injected into the capture chamber;
steps e) to h) can be carried out sequentially until all the probes have been injected into the capture chamber;
steps a), c), e) and g) can be carried out by deformation, respectively, of the liquid sample container, of the buffer solution container and of the probe container; and/or
steps a), c), e) and g) can be carried out by pressurization, respectively, of the liquid sample container, of the buffer solution container and of the probe container.

Other features of the invention will be set out in the detailed description hereinafter, given with reference to the attached figures which represent, respectively:

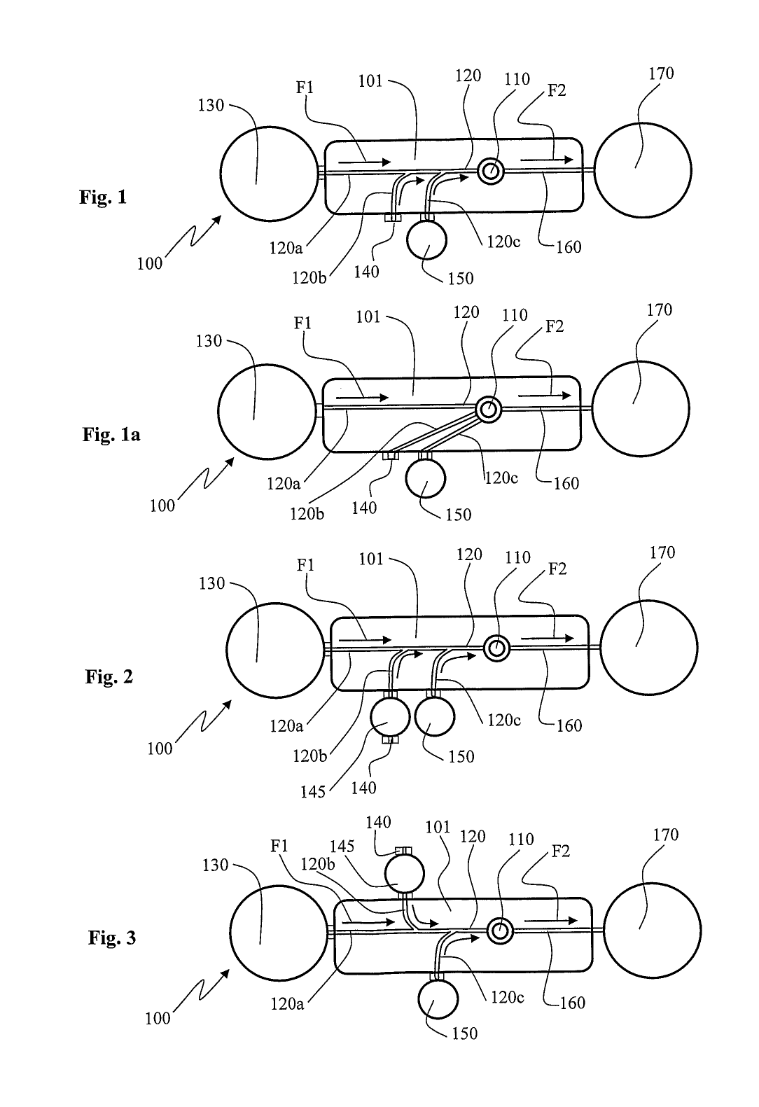

FIG. 1, a diagrammatic plan view of a first embodiment of a disposable device according to the invention;

FIG. 1a, a diagrammatic plan view of a variant of the embodiment of FIG. 1;

FIG. 2, a diagrammatic plan view of a second embodiment of a disposable device according to the invention;

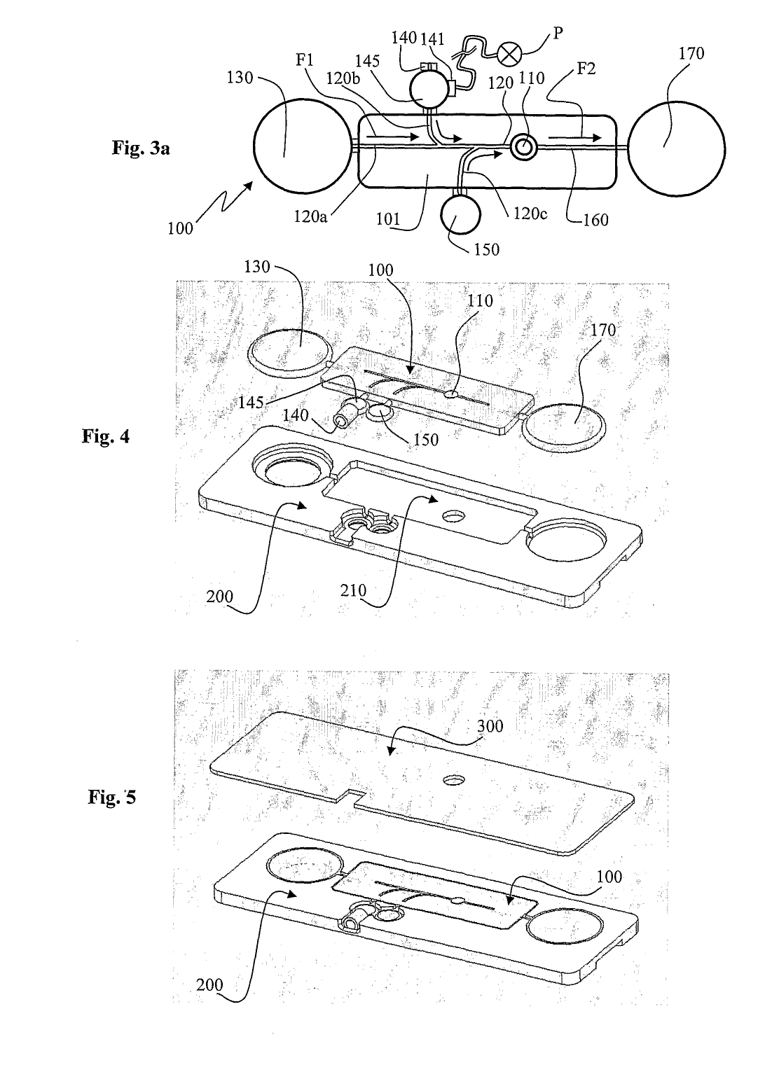

FIG. 3, a diagrammatic plan view of a third embodiment of a disposable device according to the invention;

FIG. 3a, a diagrammatic plan view of the embodiment of FIG. 3 provided with a means for sucking liquid to the liquid sample container;

FIGS. 4 to 9, diagrammatic perspective views of the implementation of a first embodiment of a detection system according to the invention; and

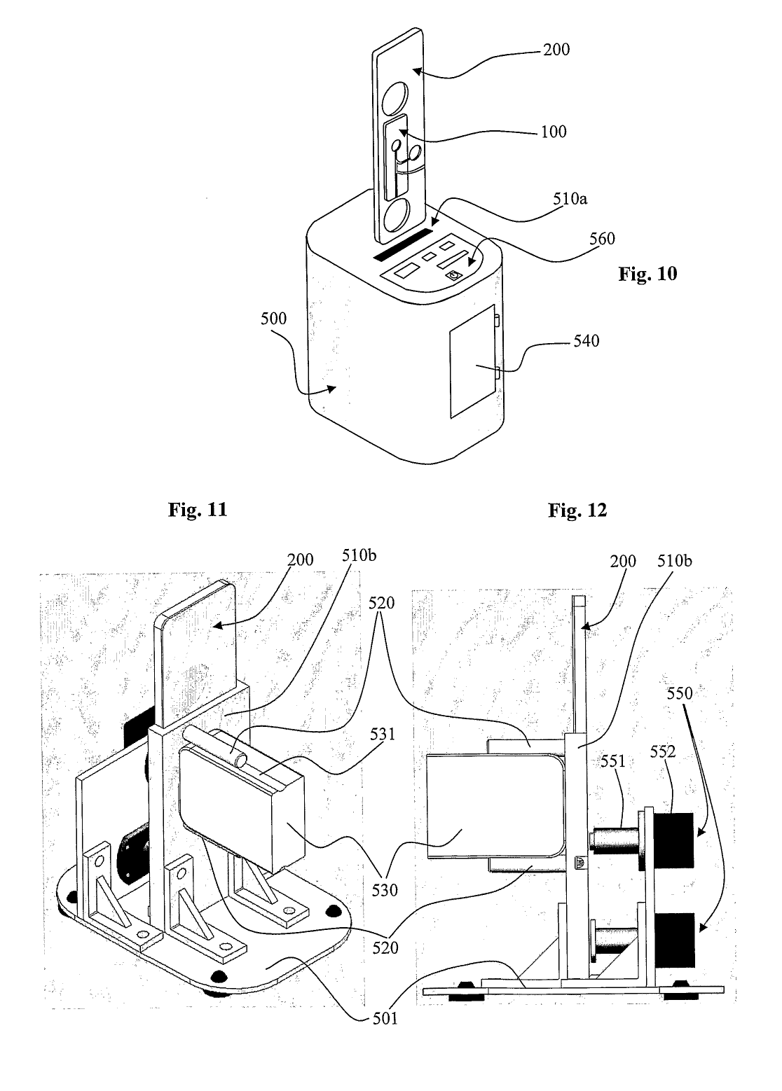

FIGS. 10 to 12, diagrammatic perspective views of the implementation of a second embodiment of a detection system according to the invention;

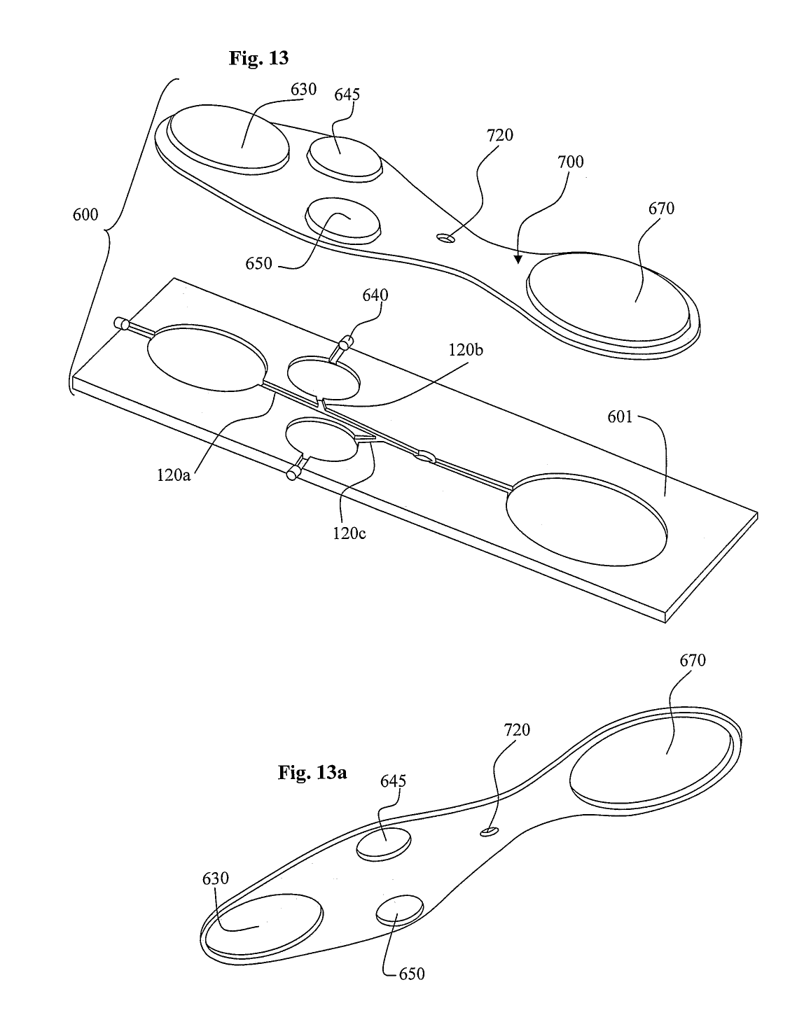

FIG. 13, a diagrammatic perspective view of a fourth embodiment of a disposable device according to the invention;

FIG. 13a, a diagrammatic perspective view of the embodiment of FIG. 13, seen from below;

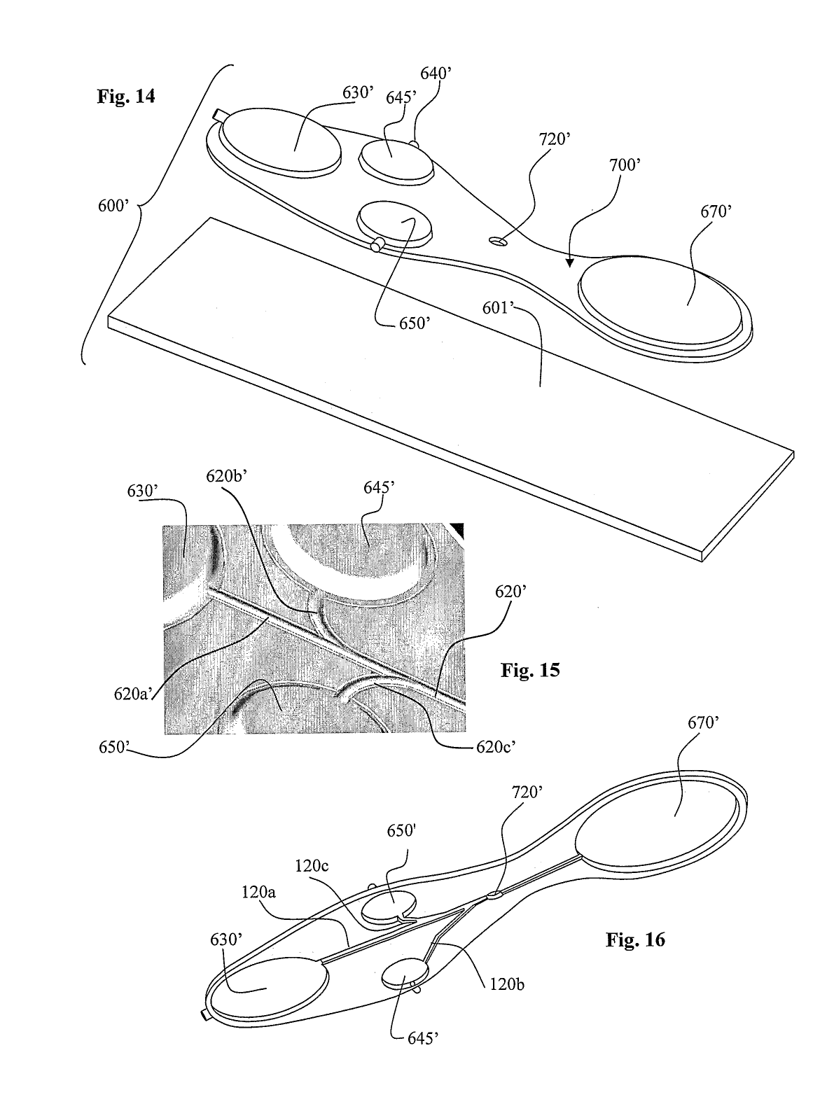

FIG. 14, a diagrammatic perspective view of a variant of the fourth embodiment of FIG. 13;

FIG. 15, a diagrammatic perspective view of an enlargement of the device of FIG. 14;

FIG. 16, a diagrammatic perspective view of the embodiment of FIG. 14, seen from below;

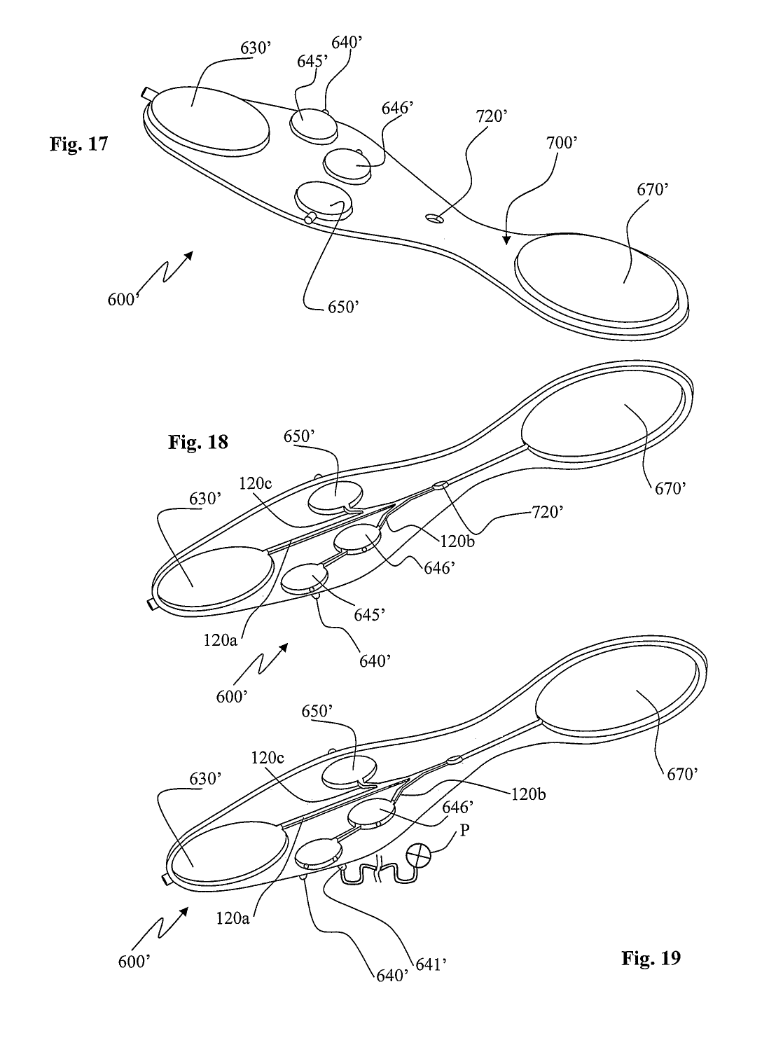

FIG. 17, a diagrammatic perspective view of a fifth embodiment of a device according to the invention and comprising a reagent container;

FIG. 18, a diagrammatic perspective view of the embodiment of FIG. 17, seen from below; and

FIG. 19, a diagrammatic perspective view of a variant of the embodiment of FIG. 17, seen from below, and comprising a means for sucking liquid to the liquid sample container.

The detection system according to the invention is illustrated by the detection of Cytomegalovirus in infants, but it can be used for the detection of any particle capable of binding specifically to a ligand bound to a capture surface and of being revealed by binding of a specific labeling probe.

The detection system according to the invention is not limited to only immunocapture reactions, but it can also be used to generate and detect other types of reactions: chemical, enzymatic, etc.

Thus, the detection system according to the invention can advantageously be used for the detection of biological entities such as bacteria, viruses, proteins, DNA or RNA strands, etc.

The detection is advantageously a detection by fluorescence.

The first embodiment of a disposable device 100 according to the invention, illustrated in FIG. 1, comprises a substrate 101 provided with a chamber 110 for capture of the particle(s) of interest to be detected. The substrate 101 comprises a fluidic channel 120 connecting, upstream relative to the direction of flow F1, the capture chamber 110 to a container pre-filled with a predefined volume of buffer solution 130, to a liquid sample injection means 140 and to a container 150 pre-filled with a predefined volume of labeling probes. These probes are included in a transporter liquid and are capable of binding specifically to the particle(s) of interest to be detected.

The substrate 101 also comprises a fluidic channel 160 connecting, downstream relative to the direction of flow F2, the capture chamber 110 to a container 170 for the recovery of the liquids that may flow, during use, from the capture chamber 110.

Advantageously, the substrate 101 is made of a material suitable for producing microfluidic channels. The characteristic sizes of these channels are of the order of a few hundred microns, but can range up to a few tens of nanometers. Lithography or etching of a material such as glass, silicon or quartz can be used to produce these channels. They can also be produced by molding or stamping of polymer materials.

The upstream fluidic channel 120 comprises three fluidic paths 120a, 120b and 120c connecting, respectively, the buffer solution container 130, the injection means 140 and the labeling probe container 150. In the embodiments of FIGS. 1 to 3, the channel 120 has a main path 120a into which the secondary paths 120b and 120c open. Alternatively, in an embodiment illustrated in FIG. 1a, the three paths 120a, 120b and 120c of the fluidic channel 120 can be independent and can each open into the capture chamber 110. Other conformations can be envisioned.

In the embodiment of FIG. 1, the disposable device comprises predefined volumes of buffer solution and of probes, determined by the volume of the containers 130 and 150. The volume of liquid sample is, for its part, determined by the user who injects this sample via the injection means 140.

The buffer solution and probe containers are prefilled during manufacture, such that the user does not have to handle the reagents. It is sufficient for said user to select the disposable device suitable for the reaction that said user wishes to carry out, to inject therein a predefined volume of the test solution, and to carry out the measurement (as subsequently described).

Advantageously, the disposable device according to the invention also comprises a liquid sample container placed downstream of the sample injection means and upstream of the capture chamber. This embodiment is illustrated in FIGS. 2 and 3.

This sample container 145 makes it possible to inject the liquid sample into the capture chamber 110 with a predetermined volume, that of the container 145. In other words, before the actual detection and the injection of the sample to the capture chamber, the user fills the container 145 with the liquid sample. For example, the user can fill the container 145 using a piston device such as a syringe (see FIG. 8).

More generally, the liquid sample injection means 140, whether it is directly connected to the upstream fluidic channel 120 or connected by means of the container 145, comprises a membrane made of a leaktight material which retains its leaktightness after having been pierced. Preferably, this material is chosen from a silicone polymer such as polydimethylsiloxane (PDMS), poly(methyl methacrylate) (PMMA), polyvinyl chloride (PVC), Tygon® (manufactured by the company Saint-Gobain), etc.

In the embodiment of FIG. 2, the injection means 140 and the probe container 150 are placed on the same side of the substrate 101. The embodiment of FIG. 3 has an injection means 140 and a probe container 150 arranged on either side of the substrate 101.

The injection means can be connected directly to a source of liquid to be sampled for analysis: for example, the gastric suction circuitry of a newborn infant. Advantageously, as represented in FIG. 3a, the device according to the invention also comprises a suction channel 141 which opens into the liquid sample container 145 and is intended to be connected to a pump P in order to suck the liquid sample from the injection means 140, which is connected to the source of liquid to be sampled, to the liquid sample container 145.

Such an arrangement is particularly advantageous since it avoids a nurse having to carry out additional actions compared with those which are currently performed. In detail, the gastric suction circuitry comprises a tube connecting the newborn infant to a bottle, which is itself connected, via a tube, to a pump P. The device according to the invention can therefore replace the bottle and allow an analysis of the gastric fluid which is simple to carry out and does not require an additional action. Of course, a non-return valve (not shown) is envisioned in order to prevent the reduced pressure generated by the pump from sucking the other liquids contained in the device to the sample container via the microfluidic channels.

Alternatively or in combination, the disposable device according to the invention can also comprise at least one reagent container placed downstream of the sample injection means 140 and upstream of the capture chamber 110 (see the description of the embodiments of FIGS. 17 to 18 by analogy). In one embodiment having a sample container 145, said at least one reagent container is placed downstream of the liquid sample container 145 and upstream of the capture chamber 110. In this case, the path 120b connects the reagent container to the main path 120a or to the capture chamber 110 if each container is connected independently to this chamber (by analogy with the embodiment of FIG. 1a). Of course, non-return valves (not shown) are envisioned in order to ensure the circulation of the various liquids to the capture chamber 110.

This reagent container can be provided with a filler end-piece, but, advantageously, the container is pre-filled with a predefined volume of reagent. This avoids the person who uses the disposable device according to the invention having to have an additional action to carry out, which can cause analysis errors when the reagent with which the user fills the container is not suitable for the analysis.

In all the embodiments of a disposable device according to the invention, the containers (130, 145 and 150), and also the insertion means 140, can be placed anywhere upstream, relative to the capture chamber 110. Nevertheless, the paths 120a, 120b and 120c will not be arranged in such a way that they open out opposite one another. This is because, in this case, a liquid ejected from a container could move toward the path which is opposite it, and not to the capture chamber 110.

Advantageously, the buffer solution container 130, the labeling probe container 150 and, for the embodiments of FIGS. 2 and 3, the sample container 145 have a structure which allows them to be pressurized. This pressurization structure allows, during use, the flow of the various liquids (buffer solution, labeling probes and, optionally, liquid sample) to the capture chamber via the fluidic channel 120.

The containers containing the buffer solution and the labeling probes are filled during manufacture of the disposable device. The container intended to collect the sample is filled by the operator.

The recovery container 170 has a volume at least equal to the sum of the storage volumes of the buffer solution container, of the secondary container of the liquid sample and of the labeling probe container.

In order to make it possible to store the volume of the various liquids, the recovery container 170 can be kept at a reduced pressure, or squashed at the start via a pressurization means which operates in the opposite direction (expansion) to the other pressurization means as said other pressurization means operate (compression of containers 130, 140, 150 via pressurization means 420; see hereinafter, with reference to FIGS. 9, 11 and 12). An alternative would consist in providing for an air escape, either via a valve, or through the use of a material porous to air but not to liquids.

The fact that the disposable device according to the invention integrates all the containers of reagents necessary for the detection of particles of interest has many advantages. Thus, each device comprises calibrated volumes of reagents (buffer solution, labeling probes and, optionally, liquid sample), which allows an optimum reaction without the user having any complex handling to perform. Moreover, these containers with a predefined volume ensure the disposable nature of each device.

The recovery container 170 makes it possible to avoid discarding the reagents (buffer solution, labeling probes and liquid sample) out of the device. In particular, it avoids providing for such a container in the detection system (see below), in which container all the reagents resulting from the detection of several disposable devices would be mixed together.

According to one preferred embodiment, the buffer solution container 130, the labeling probe container 150 and, for the embodiments of FIGS. 2 and 3, the sample container 145 are made of a material that is sufficiently flexible to be deformed by an external pressure applied to the containers. Thus, each container has a first volume, termed “storage” volume, in which all the liquid fills the container, and a second volume, termed “ejection” volume, which is less than the storage volume, and in which the liquid is ejected from the container to the capture chamber.

The capture chamber 110 has at least one surface functionalized with ligands, bound to said surface, and capable of associating specifically with the molecule(s) of interest to be detected.

The operating principle of such a capture chamber is based on a immunocapture reaction at the functionalized surface of the chamber. This surface is coated with ligands such an antibodies specific for the particles to be detected, for instance the Cytomegalovirus. Next, the liquid biological sample (blood, saliva, urine, gastric juices, etc.) is injected via the injection means 140 to the capture chamber. Thus, the sample is brought into contact with the ligand-coated surface. If particles of interest are present in the sample, they are trapped by the ligands. In the case of the Cytomegalovirus or, more generally, of biological molecules, this trapping step is carried out by immuno-capture by virtue of antibodies (the ligands). When the sample has spent a sufficient amount of time in the capture chamber it is evacuated to the liquid recovery container 170. Advantageously, the capture chamber is rinsed with buffer solution flowing from the container 130. This makes it possible to evacuate the untrapped particles. The rinsing buffer solution is then evacuated to the liquid recovery container.

After this step, the labeling probes are made to flow from their container to the capture chamber. These probes are designed to bind specifically to the particles of interest trapped by the ligands in the capture chamber 110. In the case of biological particles to be detected, the probes are preferentially composed of labeled specific antibodies. This labeling is preferably carried out by grafting fluorescent molecules onto the antibodies. Thus, if biological particles have been trapped during the preceding step, the probes bind to the trapped particles. If the opposite is true, the probes are evacuated, by rinsing with buffer solution, to the liquid recovery container 170. Other types of labeling can be used, such as magnetic labels, radioactive labels, etc.

Next, the presence or absence of the probes and therefore of the particles of interest to be detected is measured. For this purpose, the capture chamber 110 has at least one surface designed to allow this detection of the labeling probes. In the case of a detection of fluorescence, the capture chamber has a cover which is transparent to the excitation and emission wavelengths of the fluorescent particles used.

The abovementioned method for using the disposable device according to the invention can be carried out in a single step. In other words, the sample can be injected in one go, and the labeling probes can also be injected in one go.

However, preferentially, the method for using the device according to the invention is carried out sequentially. In this case, the sample is injected in several stages, between each one of which the chamber can optionally be rinsed with buffer solution. Likewise, the labeling probes can be injected sequentially, i.e. they are injected in several stages, between each of which the capture chamber is rinsed with buffer solution.

This sequential use ensures better capture of the particles of interest and therefore better sensitivity of the device.

In the embodiments of FIGS. 1 to 3, the device according to the invention has a substantially rigid substrate 101 incorporating the fluidic channels and the capture chamber. The various containers 130, 150, 170, and optionally 145, are attached to this substrate 101 and placed in fluidic connection 120 and 160. The manufacture of such a structure is very advantageous in terms of manufacturing cost when it uses the techniques of molding, hot-stamping, etc. However, it can be fragile at the level of the connections between the containers and the substrate.

For this purpose, it is envisioned to incorporate this device 100 into a removable cassette 200, as represented in FIG. 4, making it possible to rigidify the device. This removable cassette 200 comprises device-holding means 210. In the embodiment of FIG. 4, these holding means consist of an imprint 210 complementary to the device 100. Preferentially, as illustrated in FIG. 5, the cassette 200 comprises a cover 300 which holds the device 100 in the cassette 200.

In order to implement the above method, the invention proposes a detection system of which two embodiments are illustrated, respectively, in FIGS. 6 to 9 and 10 to 12.

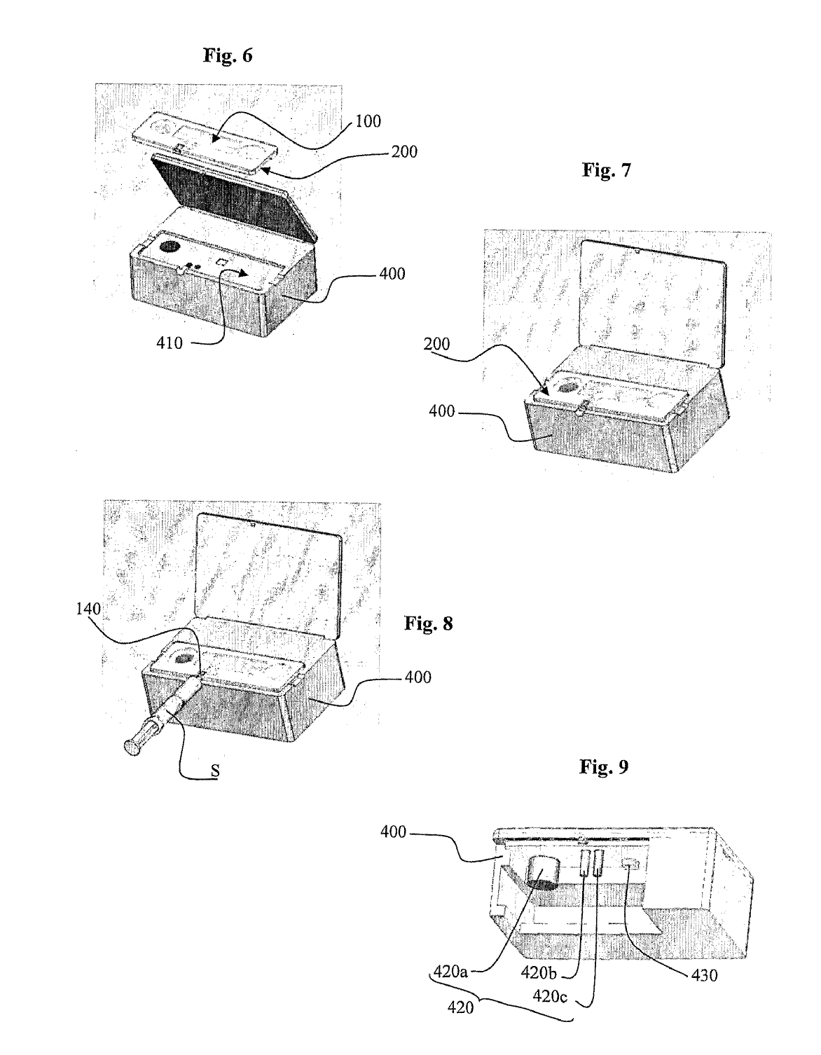

In the first embodiment, the detection system comprises a housing 400 comprising a receptacle 410 designed to receive a disposable device 100 according to the invention. In FIG. 6 this receptacle 410 consists of an imprint complementary to the cassette 200. Once the cassette 200 is placed in the receptacle 410 (FIG. 7), the sample is injected into the disposable device according to the invention via the sample injection means 140 (FIG. 8). The injection is carried out, in this example, using a syringe S.

The detection system also comprises a means for pressurization 420 of the various containers of the disposable device. In FIG. 9, the pressurization means 420 comprises a mechanism 420a for pressurization of the buffer solution container, and two mechanisms 420b and 420c for pressurization, respectively, of the sample containers and of the labeling probe container.

The detection system also comprises a means for detection 430 of the labeling probes. It is therefore understood that the removable cassette is capable of allowing pressurization of the containers of the device that it contains.

The pressurization means can be mechanical, electromechanical, pneumatic or hydraulic means, designed to deform the containers by squashing. For example, these pressurization means are pistons.

A second embodiment of a detection system according to the invention is illustrated in FIGS. 10 to 12. In this embodiment, the housing 500 comprises a reception slot 510a designed to receive a disposable device according to the invention. In the embodiment illustrated, this disposable device 100 is combined, as for FIGS. 4 and 5, with a removable cassette 200. It is therefore this cassette 200 which is inserted into the reception slot 510a. More generally, the cassette 200 can be placed horizontally or vertically and even according to any orientation required by the internal structure of the detection system.

FIGS. 11 and 12 illustrate the interior of the system of FIG. 10. Thus, this system comprises a receptacle 510b opposite the slot 510a (not illustrated on these figures). This receptacle is provided, moreover, with means for holding 520 a means for detecting 530 the labeling probes. Of course, these holding means 520 could be independent of the receptacle 510a and attached elsewhere in the system, for example to the frame 501.

Advantageously, these holding means allow removable attachment of the detection means 530. In the example illustrated, the holding means 520 are cylindrical pegs cooperating, slightly forcibly, with grooves 531 made on the detection means 530. The detection means 530 can therefore be removed from the housing via a cover 540 (FIG. 10), and replaced with another detection means.

This structure makes it possible either to replace a defective detection means or to change the type of detection means.

The detection system also comprises the means for pressurization of the various containers (buffer solution, labeling probes and, optionally, sample). In FIG. 12, these pressurization means 550 consist of pistons 551 activated by motors 552. Depending on the control program chosen by the user or on the direct commands made by the user, the pistons 551 apply a pressure to the containers and deform them, such that the liquid that they contain is ejected via the fluidic channels to the capture chamber.

An alternative to the pressure means described consists in providing for pins positioned inside the housing, in such a way that they cause sequential squashing of the containers at the time of the (manual or automatic) introduction of the cassette into the housing.

According to an alternative that is not illustrated, the buffer solution, labeling probe and, optionally, sample containers are made of a material which is sufficiently rigid to allow, during use, an internal raised pressure that is sufficient to drive the liquids (buffer solution, labeling probes and, optionally, the sample) to the capture chamber.

In order to use such a device, the detection system according to the invention comprises a means for injection of a fluid under pressure into the containers. For example, this injection means can consist of several needles connected to a pneumatic system and inserted into the containers. For this purpose, it can be envisioned that the sufficiently rigid material comprises a membrane made of a leaktight material that retains its leaktightness after having been pierced. This material can be chosen from a silicone polymer such as polydimethylsiloxane, poly-(methyl methacrylate), polyvinyl chloride or Tygon®.

In this embodiment, the method of use is carried out by pressurizing the buffer solution container, the probe container and, optionally, the liquid sample container.

In all the embodiments previously described, it is advantageously envisioned that the buffer solution container, the sample container and the labeling probe container have a pressure-sensitive non-return valve which makes it possible to open the valve when a threshold pressure is applied. This pressure can be either an external pressure or an internal pressure.

It can also be envisioned that the liquid recovery reservoir 170 is also provided with a non-return valve. In this way, the fluids evacuated cannot return to pollute the capture chamber.

A detection system according to the invention can be entirely automatic. In this case, it detects the insertion of a device according to the invention and controls, according to a preprogrammed sequence, the fluid management members (the means for pressurization of the buffer solution container, of the labeling probe container and, optionally, the sample container).

The detection system can be suitable for various cassettes 200, each suitable for a predetermined pathological condition. The detection system recognizes the pathological condition in question (reading of a barcode present on the cassette, for example) and automatically adjusts the fluid management (flow rate, timing, etc.). Thus, in the case of an automated procedure, the same detection system can be used for various pathological conditions.

However, a detection system according to the invention preferably comprises a user interface 560 (FIG. 10). In this case, the user controls the implementation of the detection. It is possible to choose from:
an automatic mode, in which the system handles the fluid management and the detection,
a semi-automatic mode, in which the user chooses the fluid management program, or
a manual mode, in which the experienced user himself controls the ejection of the fluids out of their respective container and controls the detection.

The detection system according to the invention can comprise a means for temperature control of the cassette. This is because, in the case of detection by fluorescence, if the temperature is too low, the fluorescence efficiency is low and the detection is difficult. Conversely, if the temperature is too high, the antibodies can be degraded and the capture of entities of interest may be relatively inefficient.

FIG. 13 illustrates a fourth embodiment of a disposable device 600 according to the invention. In this embodiment, the buffer solution container 630, the labeling probe container 650, the liquid recovery container 670 and, optionally, the sample container 645 and/or at least one reagent container are directly incorporated into the substrate 601 of the device. This embodiment avoids recourse to a cassette in order to rigidify the device.

This embodiment can consist in etching or molding the capture chamber 610, the fluidic channels 620, 620a, 620b and 620c, and also the containers, in a substrate, and then in placing, on the substrate thus etched or molded, a structured layer of a deformable material 700.

This layer 700 preferably has pressure-deformable structures arranged facing the various containers and the sample injection means 640.

Moreover, this layer 700 has an opening or a transparent part 720 facing the capture chamber, designed to allow, during use, the detection of the labeling probes bound to the particle(s) of interest which have associated specifically with the ligands in said capture chamber.

As shown in FIG. 13a which illustrates the layer 700 viewed from below compared with FIG. 13, the face of the layer 700 intended to be in contact with the substrate 601 does not bear any fluidic channel, but only containers. The liquids circulate in the channels borne by the substrate 601 and closed by the layer 700 when the containers of the layer 700 are pressurized.

A particularly advantageous variant is illustrated in FIGS. 14 to 16.

In this variant, all the structures (containers and fluidic channels) are molded in a layer of a deformable material 700′, whereas the substrate 601′ is not structured.

As shown in FIG. 16, which illustrates the layer 700′ viewed from below compared with FIG. 14, the face of the layer 700′ intended to be in contact with the substrate 601′ bears the fluidic channels and containers.

All that remains is then to deposit the layer 700′ on the substrate 601′ in order to obtain the device according to the invention. When the layer 700′ is in contact with the substrate 601′, the liquids can circulate in the channels borne by the layer 700′ and closed by the substrate 601′ when the containers of the layer 700′ are pressurized.

Contrary to the embodiment of FIG. 13, this variant does not require any particular alignment between the containers of the layer 700 and the channels borne by the substrate 601 since, in the embodiment of FIGS. 14 to 16, the substrate 601′ merely serves to close the channels during the association of the layer 700′ with the substrate 601′. The use is therefore simplified.

A cover 300 is then placed on the device according to the invention, by analogy with FIG. 5.

An additional seal (not shown) can be added between the layer of deformable material 700 or 700′ and the substrate 601 or 601′.

Alternatively or in combination, a disposable device according to the invention can also comprise a reagent container 646′ placed downstream of the sample injection means 640′ and upstream of the capture chamber 720′ (see FIGS. 17 to 19). In one embodiment which has a sample container 645′, said at least one reagent container 646′ is placed downstream of the liquid sample container 645′ and upstream of the capture chamber 610′. In this case, the path 120b connects the reagent container 646′ to the main path 120a or to the capture chamber 720′ if each container is independently connected to this chamber (by analogy with the embodiment of FIG. 1a). Of course, non-return valves (not shown) are provided for in order to ensure circulation of the various liquids to the capture chamber 720′.

The reagent container 646′ can be provided with a filler end-piece, but, advantageously, the container is prefilled with a predefined volume of reagent. This avoids the person who uses the disposable device according to the invention having to carry out an additional action, which can cause analysis errors when the reagent with which the user fills the container is not suitable for the analysis.

As for the embodiments of FIGS. 1 to 12, the embodiments of FIGS. 13 to 16 can also comprise an injection means directly connected to a source of liquid to be sampled for analysis: for example, the gastric suction circuitry of a newborn infant. This is illustrated in FIG. 19. Advantageously, as represented in FIG. 3a, the disposable device according to the invention comprises a suction channel 641′ which opens into the liquid sample container 645′ and is intended to be connected to a pump P in order to suck the liquid sample from the injection means 640′, which is connected to the source of liquid to be sampled (not shown), to the liquid sample container 645′. Of course, a non-return valve (not shown) is provided for in order to prevent the reduced pressure generated by the pump from sucking the other liquids contained in the device to the sample container via the microfluidic channels.

An exemplary embodiment of a device according to the invention is given hereinafter:
cassette dimensions: 185×50×6 mm
capture chamber: chromium-gold deposition by spraying onto the capture surface, then functionalization by binding of trapping antibodies (IgG type)

| | Container | External |
|---|---|---|
| Name | volume (ml) | diameter (mm) |
| Buffer solution container | 3 | 35.7 |
| Sample container | 1 | 20.6 |
| Labeling probe container | 1 | 20.6 |
| Liquid recovery container | 5.5 | 48.3 |

The containers are cylinders with a height h = 3 mm.

Many viruses are not involved in this type of detection system in the context of conventional screening. This is because, in practice, a serological test is fully sufficient, such as for enteroviruses (adenoviruses, rotaviruses, etc.), the HIV virus, the hepatitis A, B and C viruses, etc. On the other hand, in certain emergency contexts, it is necessary to have a rapid and effective means for detecting any trace of virus. Before a transplant, the graft undergoes many tests in order to be sure that it is not carrying any virus, such as retroviruses (HIV, HTLV), hepatitis viruses, herpes-type viruses, Epstein Barr virus, etc. However, the success of the transplant depends greatly on the time interval between the moment the graft is taken from the donor and the moment the graft is transplanted into the recipient. Reducing this time interval is therefore a considerable challenge, that the detection system and device according to the invention make it possible to solve.

The liquid sample used can be a pure or diluted sample of blood, saliva, urine, gastric juice, or other body fluid. It can also be prepared by dabbing a substrate (inside of the cheek, clothing, baggage, etc.) then rinsing using an inert liquid and recovering this rinsing liquid.

The device, the system and the method according to the invention will allow, for example, detection at the bedside of patients or emergency medicine in the field.

In addition, a disposable device according to the invention can be preserved as evidence or for a second assessment.

1. A disposable device for the detection of one or more particles of interest present in a liquid sample, characterized in that it comprises a substrate provided with:
a chamber for capturing the particle(s) of interest to be detected;
a fluidic channel connecting, upstream relative to the direction of flow during use, the capture chamber:
to a container pre-filled with a predefined volume of buffer solution;
to a liquid sample injection means;
to a container pre-filled with a predefined volume of labeling probes capable of binding to the particle(s) of interest to be detected; and
a fluidic channel connecting, downstream relative to the direction of flow during use, the capture chamber to a container for the recovery of the liquids that may flow, during use, from the capture chamber.

2. The disposable device as claimed in claim 1, in which the substrate also comprises a liquid sample container placed down-stream of the sample injection means and upstream of the capture chamber.

3. The disposable device as claimed in claim 1, in which the buffer solution container and the labeling probe container have a pressurization structure designed to allow the flow, during use, of the buffer solution and of the labeling probes to the capture chamber via the upstream fluidic channel.

4. The disposable device as claimed in claim 1, in which the sample container has a pressurization structure designed to allow the flow, during use, of the sample to the capture chamber via the upstream fluidic channel.

5. The disposable device as claimed in claim 3, in which the buffer solution container and the labeling probe container are made of a material that is sufficiently flexible to be deformed by an external pressure, between a storage volume and an ejection volume.

6. The disposable device as claimed in claim 4, in which the sample container is made of a material that is sufficiently flexible to be deformed by an external pressure, between a storage volume and an ejection volume.

7. The disposable device as claimed in claim 3, in which the buffer solution container and the labeling probe container are made of a material that is sufficiently rigid to allow, during use, an internal raised pressure that is sufficient to drive respectively the buffer solution and the labeling probes to the capture chamber.

8. The disposable device as claimed in claim 4, in which the sample container is made of a material that is sufficiently rigid to allow, during use, an internal raised pressure that is sufficient to drive the sample to the capture chamber.

9. The disposable device as claimed in claim 7, in which the sufficiently rigid material comprises a membrane made of a leaktight material that retains its leaktightness after having been pierced, preferably chosen from a silicone polymer, such as polydimethylsiloxane (PDMS), poly(methyl methacrylate) (PMMA), polyvinyl chloride (PVC) and Tygon®.

10. The disposable device as claimed in claim 1, in which the liquid sample injec-tion means comprises a membrane made of a leaktight material that retains its leaktightness after having been pierced, preferably chosen from a silicone polymer, such as polydimethylsiloxane (PDMS), poly(methyl methacrylate) (PMMA), polyvinyl chloride (PVC) and Tygon®.

11. The disposable device as claimed in claim 3, in which the buffer solution container, the sample container and the labeling probe container have a pressure-sensitive non-return valve which allows, during use, opening of the valve when a threshold pressure is applied.

12. The disposable device as claimed in claim 2, in which the recovery container has a volume at least equal to the sum of the volumes of the buffer solution container, of the secondary container of the liquid sample and of the labeling probe container.

13. The disposable device as claimed in claim 1, in which the capture chamber has:
at least one surface functionalized with ligands, bound to said surface, and capable of associating specifically with the particle(s) of interest to be detected; and
at least one surface designed to allow, during use, the detection of the labeling probes bound to the particle(s) of interest which have associated specifically with the ligands.

14. The disposable device as claimed in claim 1, in which the substrate also comprises at least one liquid sample reagent container placed downstream of the sample injection means and upstream of the capture chamber.

15. The disposable device as claimed in claim 2, in which the substrate also comprises at least one reagent container, placed downstream of the liquid sample container and upstream of the capture chamber.

16. The disposable device as claimed in claim 2, also comprising a suction channel which opens into the liquid sample container and which can be connected to a pump for sucking the liquid sample from the injection means to the liquid sample container.

17. A system for the detection of one or more particles of interest present in a liquid sample, wherein the system is provided with a housing comprising:
a receptacle configured to receive a disposable device as claimed in claim 1;
a means for pressurization of the container pre-filled with a predefined volume of buffer solution and of the container pre-filled with a predefined volume of labeling probes; and
a labeling probe detection means.

18. The detection system as claimed in claim 17, comprising a receptacle designed to receive a disposable device, the disposable device comprising:
a chamber for capturing the particle(s) of interest to be detected;
a fluidic channel connecting, upstream relative to the direction of flow during use, the capture chamber:
to a container pre-filled with a predefined volume of buffer solution;
to a liquid sample injection means;
to a container pre-filled with a predefined volume of labeling probes capable of binding to the particle(s) of interest to be detected; and
a fluidic channel connecting, downstream relative to the direction of flow during use, the capture chamber to a container for the recovery of the liquids that may flow, during use, from the capture chamber, and wherein the substrate also comprises a liquid sample container placed down-stream of the sample injection means and upstream of the capture chamber and also comprising a means for pressurization of the sample container.

19. The detection system as claimed in claim 17, comprising a receptacle designed to receive a disposable device, the disposable device comprising:
a chamber for capturing the particle(s) of interest to be detected;
a fluidic channel connecting, upstream relative to the direction of flow during use, the capture chamber:
to a container pre-filled with a predefined volume of buffer solution;
to a liquid sample injection means;
to a container pre-filled with a predefined volume of labeling probes capable of binding to the particle(s) of interest to be detected; and
a fluidic channel connecting, downstream relative to the direction of flow during use, the capture chamber to a container for the recovery of the liquids that may flow, during use, from the capture chamber, and in which the buffer solution container and the labeling probe container have a pressurization structure designed to allow the flow, during use, of the buffer solution and of the labeling probes to the capture chamber via the upstream fluidic channel, wherein the buffer solution container and the labeling probe container are made of a material that is sufficiently flexible to be deformed by an external pressure, between a storage volume and an ejection volume, and in which the pressurization means is a mechanical, electromechanical, pneumatic or hydraulic device designed to deform the containers.

20. The detection system as claimed in claim 17, comprising a receptacle designed to receive a disposable device, the disposable device comprising:
a chamber for capturing the particle(s) of interest to be detected;
a fluidic channel connecting, upstream relative to the direction of flow during use, the capture chamber:
to a container pre-filled with a predefined volume of buffer solution;
to a liquid sample injection means;
to a container pre-filled with a predefined volume of labeling probes capable of binding to the particle(s) of interest to be detected; and
a fluidic channel connecting, downstream relative to the direction of flow during use, the capture chamber to a container for the recovery of the liquids that may flow, during use, from the capture chamber, and in which the buffer solution container and the labeling probe container have a pressurization structure designed to allow the flow, during use, of the buffer solution and of the labeling probes to the capture chamber via the upstream fluidic channel, wherein the buffer solution container and the labeling probe container are made of a material that is sufficiently rigid to allow, during use, an internal raised pressure that is sufficient to drive respectively the buffer solution and the labeling probes to the capture chamber, and in which the pressurization means comprises a means for injecting a fluid under pressure into the containers.

21. The detection system as claimed in claim 17, also comprising a removable cassette designed to receive the substrate of a disposable device as claimed in any one of the preceding claims, and intended to be inserted into the receptacle.

22. A method for using a disposable device as claimed in claim 1, wherein the method comprises the following steps:
a) injecting the sample to the capture chamber, in order to trap the particles of interest to be detected;
b) evacuating the sample to the liquid recovery container;
c) causing the buffer solution to flow from its container to the capture chamber, in order to rinse the untrapped particles from said chamber;
d) evacuating the buffer solution to the liquid recovery container;
e) causing the probes to flow from their container to the capture chamber, so that they bind to the particles of interest trapped in the capture chamber;
f) evacuating the probes to the liquid recovery container;
g) causing the buffer solution to flow from its container to the capture chamber, in order to rinse the unbound probes from said chamber;
h) evacuating the buffer solution to the liquid recovery container;
i) measuring the presence or absence of the probes, and therefore of the particles of interest to be detected.

23. The method as claimed in claim 22, in which steps a) to d) are carried out sequentially until all of the sample has been injected into the capture chamber.

24. The method as claimed in claim 22, in which steps e) to h) are carried out sequentially until all the probes have been injected into the capture chamber.

25. The method as claimed in claim 22, in which steps a), c), e) and g) are carried out by deformation, respectively, of the liquid sample container, of the buffer solution container and of the probe container.

26. The method as claimed in claim 22, in which steps a), c), e) and g) are carried out by pressurization, respectively, of the liquid sample container, of the buffer solution container and of the probe container.
